(19) United States
(12) Patent Application Publication
(10) Pub. No.: US 2018/0264242 A1
(43) Pub. Date: Sep. 20, 2018

(54) DEVICE FOR RELEASING A PHARMACEUTICAL ONTO THE SKIN OF A PATIENT

(71) Applicant: AM NOD Inc., East Meadow, NY (US)

(72) Inventors: Andrew HOFFMAN, East Meadow, NY (US); Martin KIRBY, Fountain Valeey, CA (US); Aaron GRAY, Fountain Valley, CA (US); Doug WALKER, Fountain Valley, CA (US); Craig BROMAN, Fountain Valley, CA (US); Derek BISSELL, Fountain Valley, CA (US)

(73) Assignee: AM NOD Inc., East Meadow, NY (US)

(21) Appl. No.: 15/886,984

(22) Filed: Feb. 2, 2018

Related U.S. Application Data
(60) Provisional application No. 62/462,599, filed on Mar. 16, 2017.

(51) Int. Cl. A61M 35/00

(52) U.S. Cl. CPC A61M 35/00; A61M 2205/50; A61M 2230/06; A61M 2205/3569; A61M 2205/581; A61M 2205/18; A61M 2205/8206; A61M 2205/502

(57) ABSTRACT

A wearable device for administering a pharmaceutical has a housing, a heart monitor disposed in the housing, a microprocessor connected to the heart monitor, a receptacle for holding a pharmaceutical disposed in the housing, the receptacle being in communication with a conduit leading out of the housing, an actuator connected to the receptacle and being configured to release the contents of the receptacle into the conduit upon a command from the microprocessor, and a power source disposed in the base body and being configured to power the microprocessor, the heart monitor and the actuator. The microprocessor is programmed to actuate the actuator to release the contents of the receptacle when a heart rate measured by the heart monitor falls outside a preprogrammed range.

### CROSS-REFERENCE TO RELATED APPLICATIONS

This application claims priority under 35 USC 119(e) of U.S. Provisional Application No. 62/462,599, filed on Mar. 16, 2017, the disclosure of which is herein incorporated by reference.

### BACKGROUND OF THE INVENTION

### 1. Field of the Invention

This invention relates to a device for releasing a pharmaceutical substance onto the skin of patient. In particular, the invention relates to a wristband device that monitors the patient's heartbeat and releases the pharmaceutical when the heartbeat goes outside a designated range.

### 2. The Prior Art

When a person has overdosed on narcotics, an opioid antagonist such as naloxone (Narcan®) is often administered to counteract the effects of the narcotic. However, by the time it has been determined that such an opioid antagonist is necessary, it is often too late, and the person has died. Naloxone is often administered by injection or inhalation, which requires the cooperation of the victim or assistance from a third party. It would be desirable to develop a system where the opioid antagonist can be delivered to the person automatically when needed.

### SUMMARY OF THE INVENTION

It is an object of the invention to provide a device for automatically administering Naxolone or other pharmaceutical to a patient in need. It is another object of the invention to provide a device that monitors the patient's heartbeat and automatically administers the pharmaceutical when the heartbeat is outside of a prescribed range. It is another object of the invention to provide a device for administering a pharmaceutical that is simple to use and easy to refill.

These and other objects are accomplished by a wearable device for administering a pharmaceutical in which the device is formed from a base body having a housing, a heart monitor, a microprocessor connected to the heart monitor, a receptacle for holding a pharmaceutical, the receptacle being in communication with a conduit leading out of the housing, an actuator connected to the receptacle and being configured to release the contents of the receptacle into the conduit upon a command from the microprocessor, and a power source disposed in the base body and being configured to power the microprocessor, the heart monitor and the actuator. The microprocessor is programmed to actuate the actuator to release the contents of the receptacle when a heart rate measured by the heart monitor falls outside a preprogrammed range. For example, if the medication is an opioid antagonist such as Naloxone, when the heart rate falls below a prescribed level, the actuator acts to release the medication to counteract the reduced heart rate caused by an opioid overdose.

The power source is preferably a battery, which can be either disposable or rechargeable. A charging port can be disposed in the housing for inserting a charging plug if the battery is rechargeable.

In one embodiment, a wrist band is connected to the housing so that the device can be worn on a wrist of a user. The wrist band can be lockable or removable depending on the prescribed use (i.e., mandatory or voluntary).

So that the medication does not seep out and evaporate or drip off of the user, the device has an absorbent pad located on a bottom surface of the housing and in communication with the conduit. This way, when the medication is released from the receptacle, the contents of the receptacle are released onto the absorbent pad and held there for an extended period of time, so that the medication can be absorbed transdermally.

In another embodiment, a seal is formed on a bottom surface of the base body and enclosing an area on the bottom surface of the base body. The seal acts to seal off this area when the device is worn by a user, so that medication released into the area remains in that area only and does not leak or run off on the user's skin.

In a preferred embodiment, the receptacle is removable from the housing so that the pharmaceutical can be refilled and/or replaced. Preferably, the receptacle is contained in a cartridge that is easily snapped into and out of the housing. The entire cartridge itself can be disposable and replaceable, to make medication changes and refills simple.

In another preferred embodiment, both the receptacle and power source are disposed in a removable cartridge, so that when the medication is replaced, the power source, usually in the form of a battery, is replaced as well.

In one embodiment, the actuator is comprised of a plunger connected to a solenoid valve. The plunger is disposed in an opening in the receptacle, so that actuation of the solenoid valve by the microprocessor depresses the plunger which unseals the opening and releases the medication out of the receptacle. The microprocessor can be programmed to actuate the solenoid valve for only a predetermined amount of time, so that only a pre-set dosage of the medication is released. Then if the heart rate does not return to normal after a certain amount of time, the microprocessor can actuate the valve again, to release a second dose. This can be repeated at varying dosages until the heart rate is returned to a normal range, or the medication is entirely depleted.

In one embodiment, the housing has a display connected to the microprocessor, the display being configured for displaying results of the heart monitor, the time of day, and any other desired information.

In another embodiment, there can be an audible alarm connected to the microprocessor, the alarm being triggered when the heart rate falls out of the preprogrammed range. Thus, along with release of medication, the alarm can alert third parties of a dangerous situation. In a further embodiment, there can be a transmitter connected to the microprocessor, the transmitter sending an alert to a remote receiver when the heart rate falls out of the preprogrammed range. In yet a further embodiment, there can be a global positioning system (GPS) connected to the microprocessor, so along with the alert send by the transmitter, the user's GPS coordinates are also sent so that third parties can locate the user when an alert signal is sent out.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a device according to the invention;

FIG. 2 shows a bottom view of the device of FIG. 1;

FIG. 3 shows a side cross-sectional view of the components in the device according to the invention;

FIG. 4 shows a block diagram of the components in the device according to the invention;

FIG. 5 shows an alternative embodiment of device according to the invention;

FIG. 6 shows another alternative embodiment of the device according to the invention; and

FIG. 7 shows the interior of the device according to FIG. 6 with the cartridge removed.

### DETAILED DESCRIPTION OF THE PREFERRED EMBODIMENTS

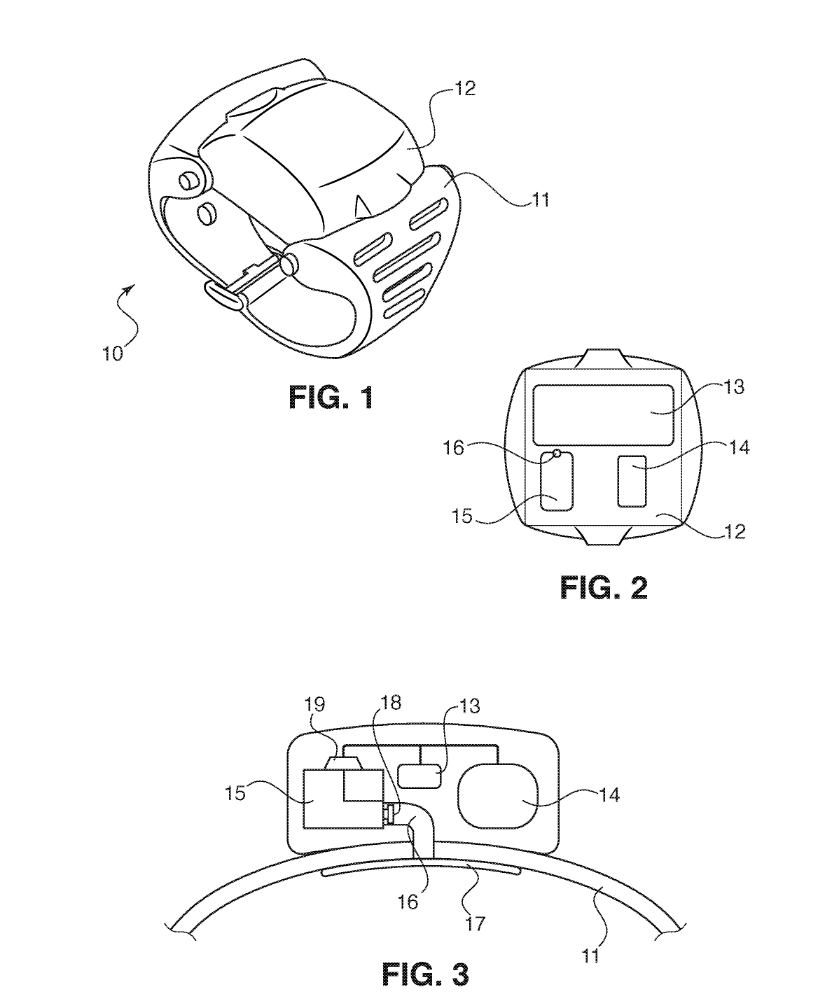

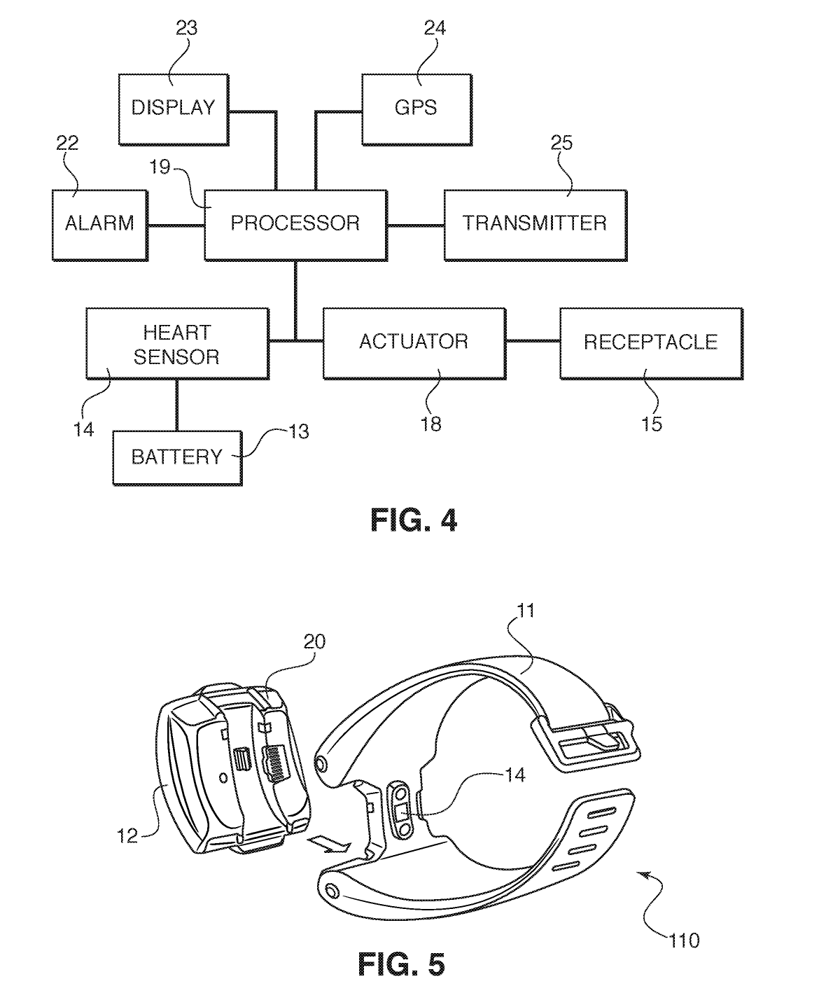

Referring now in detail to the drawings and, in particular, FIGS. 1-4 show one embodiment of the device according to the invention. Device 10 comprises a wrist band 11 and a housing 12, which holds a battery 13, a heart rate monitor 14 and a pharmaceutical receptacle 15 having a conduit 16 to the outside. FIG. 4 shows an interior cross-sectional view, in which heart rate monitor 14 is connected to a microprocessor 19, which in turn is connected to actuator 18 in the form of a plunger mechanism. When the heart rate drops below a threshold level, actuator 18 in housing 12 causes a dosage of the pharmaceutical to be released out of conduit 16 onto the skin of the user for transdermal absorption. An absorbent pad 17 can be disposed on the bottom surface of device 10 to absorb the pharmaceutical and maintain its contact with the skin without risk of the pharmaceutical dripping off or evaporating.

The components of the invention are shown schematically in FIG. 4. While several of the components are optional, all embodiments of the device comprise a power source (here in the form of a battery 13 connected to a heart sensor 14, a microprocessor 19 and an actuator 18, which upon command from microprocessor 19, releases the contents of receptacle 15 when the heart sensor 14 senses a heart rate outside a desired range. Also connected to microprocessor 19 can be an audible alarm 22, which can sound at the same time that actuator 18 is releasing the contents of receptacle 15 to alert third parties of a dangerous situation. In addition, a display 23 can be disposed on the housing, for displaying the current heart rate as measured by the heart sensor, as well as any other desired information. A transmitter 25 can be connected to microprocessor 19 to send an alert to a remote receiver when the measured heart rate falls outside the prescribed range. In addition, a GPS module 24 can be disposed in the housing and the coordinates of the device can be sent with the alert, so that third parties can locate the device in an urgent situation.

An alternative embodiment of the device 10 is shown in FIG. 5. Here, housing 12 is removable from wrist band 11, and can hold the battery 13 (not shown), and the receptacle 15, which can be replaced as needed. The heart sensor 14 is embedded in the wrist band, along with the microprocessor. Once housing 12 is connected, the device works the same as described above.

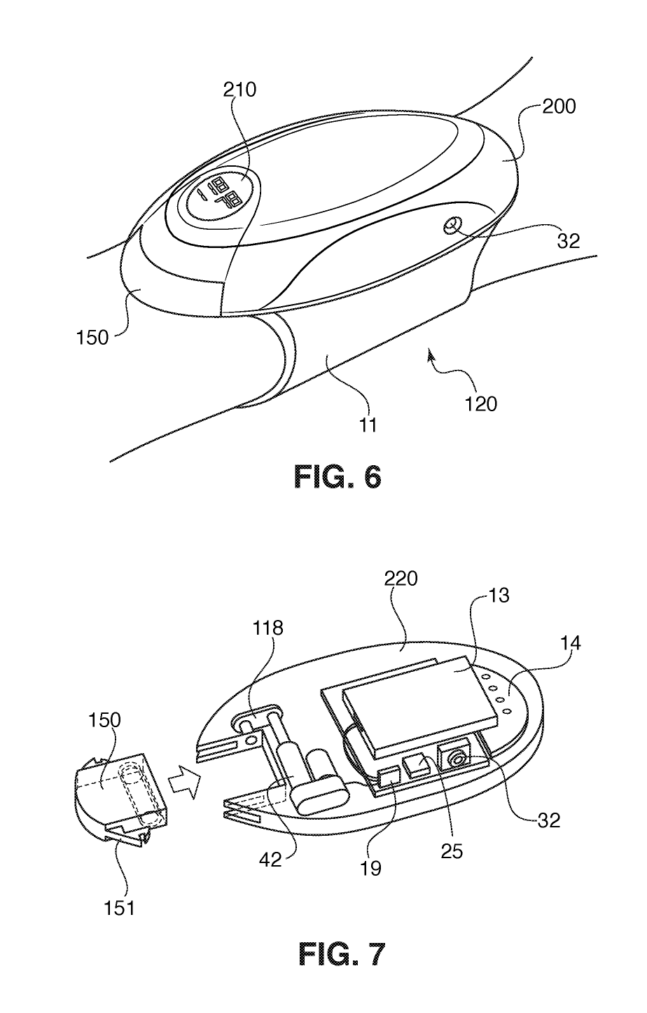

FIGS. 6 and 7 show another alternative embodiment of the invention. Here, device 120 also has a wrist band 11 connected to a housing 200, on which a display 210 is disposed for displaying the heart rage of the user, as well as the time. The housing has a base plate 220 on which battery 13 is disposed. Battery 13 can be disposable or can be a reachargable by connection of a wire through charging port 32. Battery 13 is connected to a heart sensor 14, microprocessor 19, transmitter 25 for transmitting alerts to a remote receiver. Microprocessor 19 is also connected to solenoid valve 42, which actuates plunger 118 if the heart rate sensed by sensor 14 falls below a pre-programmed heart rate. As shown in FIG. 17, receptacle 150 is removable from housing 200 for refilling. Receptacle 150 can be snapped into base plate 220 using latches 151.

The present invention provides a convenient and safe way to administer medication, especially opioid antagonists in an emergency situation, without needing medical personnel present and without requiring participation from the user, who may be incapacitated.

Accordingly, while only a few embodiments of the present invention have been shown and described, it is obvious that many changes and modifications may be made thereunto without departing from the spirit and scope of the invention.

What is claimed is:

1. A wearable device for administering a pharmaceutical, comprising:
a heart monitor;
a microprocessor connected to the heart monitor;
a receptacle for holding a pharmaceutical, the receptacle being in communication with a conduit leading out of the device;
an actuator connected to the receptacle and to the microprocessor and being configured to release the contents of the receptacle into the conduit upon a command from the microprocessor;
a power source connected to and configured to power the microprocessor, the heart monitor and the actuator; and
a housing holding at least one of the heart monitor, receptacle and power source;
wherein the microprocessor is programmed to actuate the actuator to release the contents of the receptacle when a heart rate measured by the heart monitor falls outside a preprogrammed range.

2. The device according to claim 1, further comprising a wrist band connected to the housing so that the device can be worn on a wrist of a user.

3. The device according to claim 1, further comprising an absorbent pad located on a bottom surface of the device and in communication with the conduit, such that contents of the receptacle are released onto the absorbent pad.

4. The device according to claim 1, wherein the receptacle is removable from the device.

5. The device according to claim 4, wherein the receptacle and power source are disposed in a removable cartridge.

6. The device according to claim 1, wherein the actuator is comprised of a plunger connected to a solenoid valve, wherein actuation of the solenoid valve by the microprocessor depresses the plunger and creates an opening in the receptacle for release of the contents.

7. The device according to claim 1, wherein the device has a display connected to the microprocessor, the display being configured for displaying results of the heart monitor.

8. The device according to claim 1, wherein the pharmaceutical is Naloxone.

9. The device according to claim 1, further comprising an audible alarm connected to the microprocessor, the alarm being triggered when the heart rate falls out of the preprogrammed range.

10. The device according to claim 1, wherein the power source is a battery.

11. The device according to claim 1, wherein the battery is rechargeable.

12. The device according to claim 1, further comprising a transmitter connected to the microprocessor, the transmitter sending an alert to a remote receiver when the heart rate falls out of the preprogrammed range.

13. The device according to claim 13, further comprising a global positioning system (GPS) connected to the microprocessor.

14. The device according to claim 1, wherein the microprocessor is configured to actuate the actuator after a first actuation if the measured heart rate does not return to the preprogrammed range within a predetermined period of time.
